(19) United States
(12) Patent Application Publication
(10) Pub. No.: US 2022/0370317 A1
(43) Pub. Date: Nov. 24, 2022

(54) COMPOSITION FOR SKIN WHITENING, COMPRISING CARVONE OR SALT THEREOF AS ACTIVE INGREDIENT

(71) Applicant: BOTANICSENS, Jeonju-si, Jeollabuk-do (KR)

(72) Inventor: Tae Sun PARK, Seoul (KR)

(73) Assignee: BOTANICSENS, Jeonju-si, Jeollabuk-do (KR)

(21) Appl. No.: 17/619,641

(22) PCT Filed: Jun. 16, 2020
(86) PCT No.: PCT/KR2020/007779

§ 371 Date: Dec. 16, 2021

(30) Foreign Application Priority Data
Jun. 17, 2019 (KR) 10-2019-0071589

(51) Int. Cl. A61K 8/35; A61K 8/02; A61K 8/11; A61Q 19/02

(52) U.S. Cl. CPC A61K 8/35; A61K 8/0225; A61K 8/11; A61Q 19/02

(57) ABSTRACT

A composition containing carvone or a salt thereof as an active ingredient is disclosed. A new use of the composition is also disclosed. The composition is effective in whitening skin of a subject, and capable of exhibiting an excellent melanogenesis inhibitory effect. The composition has an excellent effect of inhibiting melanogenesis induced by melanocyte-stimulating hormone (αMSH), is safe for the human body, and has no or minimum adverse effects. Thus, the composition may be variously used as a material for cosmetics, functional health foods, food ingredients or additives, dietary supplement, nutraceutical composition, pharmaceuticals, quasi-drugs, or the like.

### TECHNICAL FIELD

This application claims priority to Korea Patent Application No. 10-2019-0071589 filed on Jun. 17, 2019, which is incorporated herein by reference in its entirety.

The present disclosure relates to a composition for skin whitening containing carvone or a salt thereof as an active ingredient, and more particularly, to a composition for skin whitening capable of exhibiting an excellent melanogenesis inhibitory effect by containing carvone or a salt thereof as an active ingredient.

### BACKGROUND ART

Human skin color is determined by the amounts of melanin, carotene and hemoglobin. Among them, melanin acts as a major factor that determines skin color. It is known that melanin is produced in melanocytes present in the epidermis of the skin and then migrates to the surrounding keratinocytes, thus indicating skin color. In addition, melanin functions to protect the skin by preventing the skin from being damaged by ultraviolet rays and removing reactive oxygen species (ROS). However, it is known that chronic UV exposure induces pigmentation by promoting melanogenesis in melanocytes.

Melanogenesis is initiated in melanosomes in melanocytes. It is known that, when the skin is exposed to external stress such as ultraviolet rays, the production of α-melanocyte stimulating hormone (α-MSH) in melanocytes increases, and α-MSH activates adenylyl cyclase by binding to melanocortin 1 receptor (MC1R), which is a membrane receptor expressed only in melanocytes, thereby increasing intracellular cyclic adenosine monophosphate (cAMP). When the intracellular cAMP level increases, cAMP response element binding protein (CREB) is phosphorylated by activation of protein kinase A (PKA), and as a result, the expression of microphthalmia transcription factor (MITF) increases. It is well known that MITF is an important transcription factor of the melanin synthesis process, and increases the level of tyrosinase which is a major enzyme of melanin synthesis, and tyrosinase acts in the initial stage of melanin synthesis to regulate the rate of melanin synthesis. In this way, the synthesis of melanin in melanocytes occurs through a complex process in which enzymes such as MITF and tyrosinase are involved.

The present inventor has conducted studies on a material having an excellent skin whitening effect, and as a result, has found that carvone or a salt thereof has an excellent melanogenesis inhibitory effect, thereby completing the present disclosure.

### PRIOR ART DOCUMENTS

Korean Patent Application Publication No. 10-2018-0117252

### DISCLOSURE

### Technical Problem

An object of the present disclosure is to provide a cosmetic composition for skin whitening containing carvone or a cosmetically acceptable salt thereof as an active ingredient.

Another object of the present disclosure is to provide a health functional food composition for skin whitening containing carvone or a food-acceptable salt thereof as an active ingredient.

Still another object of the present disclosure is to provide a pharmaceutical composition for preventing or treating skin pigmentation containing carvone or a pharmaceutically acceptable salt thereof as an active ingredient.

Yet another object of the present disclosure is to provide a quasi-drug composition for preventing or ameliorating skin pigmentation containing carvone or a pharmaceutically acceptable salt thereof as an active ingredient.

However, technical problems to be achieved by the present disclosure are not limited to the above-mentioned problems, and other problems not mentioned herein will be clearly understood by those skilled in the art from the following description.

### Technical Solution

The present disclosure provides a cosmetic composition for skin whitening containing carvone or a cosmetically acceptable salt thereof as an active ingredient.

The active ingredient may inhibit the expression of microphthalmia transcription factor (MITF) or tyrosinase.

The active ingredient may inhibit melanogenesis induced by melanocyte-stimulating hormone (α-MSH).

The composition may be prepared into any one formulation selected from the group consisting of a skin lotion, a skin softener, a skin toner, an astringent, lotion, a milk lotion, a moisturizing lotion, a nourishing lotion, a massage cream, a nourishing cream, a moisturizing cream, a hand cream, an essence, a pack, a mask pack, a mask sheet, an exfoliant, soap, shampoo, a cleansing foam, a cleansing lotion, a cleansing cream, a body lotion, a body cleanser, an emulsion, a pressed powder, a loose powder, and an eye shadow.

The active ingredient may be contained in an amount of 0.0001 to 20 wt % based on the total weight of the cosmetic composition.

The present disclosure also provides a health functional food composition for skin whitening containing carvone or a food-acceptable salt thereof as an active ingredient.

The composition may be prepared into any one formulation selected from the group consisting of tablets, granules, powders, capsules, liquid solutions, and pills.

The present disclosure also provides a pharmaceutical composition for preventing or treating skin pigmentation containing carvone or a pharmaceutically acceptable salt thereof as an active ingredient.

The present disclosure also provides a quasi-drug composition for preventing or ameliorating skin pigmentation containing carvone or a pharmaceutically acceptable salt thereof as an active ingredient.

The present disclosure also provides a method for preventing or treating skin pigmentation, comprising administering or taking a composition containing a carvone or a pharmaceutically acceptable salt thereof as an active ingredient to a subject.

The present disclosure also provides the use of a composition containing a carvone or a pharmaceutically acceptable salt thereof as an active ingredient for preventing or treating skin pigmentation.

### Advantageous Effects

The composition containing carvone or a salt thereof as an active ingredient according to the present disclosure has an excellent effect of inhibiting melanogenesis induced by melanocyte-stimulating hormone (αMSH), is safe for the human body, and has few side effects. Thus, the composition may be used variously as a material for cosmetics, health functional foods, pharmaceuticals, or quasi-drugs.

### BRIEF DESCRIPTION OF DRAWINGS

FIG. 1 shows the results of analyzing the change in the pellet color of B6F10 melanocytes treated with carbon.

FIG. 2 is a graph showing the change in melanin production in B6F10 melanocytes treated with carvone (each value is the mean±SEM of three independent experiments) [one-way ANOVA].

FIG. 3 is a graph showing the change in gene expression in B6F10 melanocytes treated with carvone (each value is the mean±SEM of three independent experiments; the letters on the bars indicate statistically significant differences at P<0.05) [one-way ANOVA].

### BEST MODE

The present inventor has found that carvone or a salt thereof exhibits a remarkably excellent whitening effect, thereby completing the present disclosure.

Hereinafter, the present disclosure will be described in detail.

The present disclosure provides a composition for skin whitening containing carvone or a salt thereof as an active ingredient.

The active ingredient may inhibit the expression of microphthalmia transcription factor (MITF) or tyrosinase. In addition, the active ingredient may inhibit melanogenesis induced by melanocyte-stimulating hormone (α-MSH).

Carvone is a volatile terpenoid-based compound. The structural formula of carbon is C₁₀H₁₄O, the molecular weight thereof is 150.2 g/mol, and the molecular structure thereof is as shown in the following Formula 1. Carvone is a pale yellow to colorless liquid with a unique minty spearmint scent of peppermint plants, and is well soluble in water and ethanol. Carvone is mainly used as a flavoring agent for air fresheners, cleansing agents, detergent, etc., and is registered as a material usable as a flavoring agent in the Food and Drug Administration (FDA) food additive database. In addition, carvone has been approved as a safe flavoring agent and a safe food additive by FEMA (Flavor and Extract Manufacturers Association) and JECFA (Joint FAO/WHO Expert Committee on Food Additives), respectively, and is commercially used for flavoring purposes. However, until now, the whitening effect of carvone has not been known. The LD50 value of carvone was reported to be 1,640 mg/kg in the oral toxicity test on rats, and was reported to be 4,000 mg/kg or higher in the transdermal toxicity test.

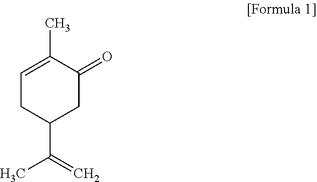

The carvone of the present disclosure may have a structure represented by Formula 1 above, more preferably a structure represented by the following Formula 1-1.

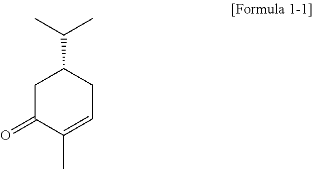

The method for obtaining the carvone of the present disclosure is not particularly limited, and carvone that is used in the present disclosure may be isolated from a natural product, or be chemically synthesized using a known method, or is commercially available.

In the present disclosure, the carvone may include a carvone hydrate, a carvone derivative, etc. which has the same efficacy as the carvone, and may also include a solvate or stereoisomer thereof.

In the present disclosure, the term “skin whitening” means not only brightening the skin tone by inhibiting the synthesis of melanin pigment, but also ameliorating skin hyperpigmentation such as spots or freckles caused by ultraviolet rays, hormones or heredity.

In the present disclosure, the term “cosmetically acceptable salt”, “food-acceptable salt”, “pharmaceutically acceptable salt” or “salt thereof” may refer to a salt form in which carvone is bound to another substance and which may exhibit activity similar to that of carvone. The “cosmetically acceptable salt”, “food-acceptable salt”, “pharmaceutically acceptable salt” or “salt thereof” may be an acid addition salt formed by a free acid. The acid addition salt may be prepared using a conventional method, for example, by dissolving a compound in an excess amount of an aqueous acid solution, and precipitating the resulting salt using a water-miscible organic solvent, for example, methanol, ethanol, acetone, or acetonitrile. In addition, the acid addition salt may be prepared by heating a compound and acid or alcohol in water (e.g., glycol monomethyl ether) in the same equimolar amounts, drying the resulting mixture by evaporation, or suction-filtering the precipitated salt.

The free acid may be an inorganic acid or an organic acid. Non-limiting examples of the inorganic acid include hydrochloric acid, phosphoric acid, sulfuric acid, nitric acid, and tartaric acid, and these inorganic acids may be used alone or in combination of two or more thereof. Non-limiting examples of the organic acid include methanesulfonic acid, p-toluenesulfonic acid, acetic acid, trifluoroacetic acid, maleic acid, succinic acid, oxalic acid, benzoic acid, tartaric acid, fumaric acid, mandelic acid, propionic acid, citric acid, lactic acid, glycollic acid, gluconic acid, galacturonic acid, glutamic acid, glutaric acid, glucuronic acid, aspartic acid, ascorbic acid, carbonic acid, vanillic acid, and hydroiodic acid. These organic acids may be used alone or in combination of two or more thereof.

In addition, the carvone may be prepared into a cosmetically acceptable or food-acceptable metal salt using a base. An alkali metal or alkali earth metal salt may be obtained, for example, by dissolving a compound in an excess amount of an alkali metal hydroxide or alkali earth metal hydroxide solution, filtering the non-soluble compound salt, and then evaporating and drying the filtrate. As the metal salt, in particular, a sodium, potassium or calcium salt may be prepared, but the present disclosure is not limited thereto. In addition, the corresponding silver salt may be obtained by reacting the alkali metal or alkali earth metal salt with an appropriate silver salt (e.g., silver nitrate).

Unless otherwise indicated, the salt of the carvone may include all salts of acidic or basic groups which may be present in compounds of the carvone. Examples of the salt of the carvone include sodium, calcium and potassium salts of a hydroxyl group, examples of other cosmetically acceptable salts of amino groups include hydrobromides, sulfates, hydrogen sulfates, phosphates, hydrogen phosphates, dihydrogen phosphates, acetates, succinate, citrates, tartrates, lactate, mandelate, methanesulfonate (mesylate), and p-toluenesulfonate (tosylate), and these salts may be prepared using a salt preparation method known in the art.

The present disclosure provides a cosmetic composition for skin whitening containing carvone or a cosmetically acceptable salt thereof as an active ingredient.

In the cosmetic composition of the present disclosure, the effective content of the active ingredient is not particularly limited, and may be about 0.0001 wt % to about 20 wt % based on the total weight of the composition. If the content of the active ingredient in the cosmetic composition is less than 0.0001 wt %, the active ingredient may not exhibit a desired skin whitening effect because the amount thereof is very small. On the other hand, if the content of the active ingredient is more than 20 wt %, the change in the effect of the active ingredient may be insignificant or the active ingredient may exhibit previously known toxicity.

The term “cosmetic composition” as used in the present disclosure refers to a composition containing the compound and having any type of formulation. Examples of formulations of cosmetics prepared using the composition include creams such as nourishing cream, eye cream, massage cream, and cleansing creams; packs; lotions such as nourishing lotion; essences; tonics such as skin softeners and nourishing tonics; powders; foundations; and makeup bases. To achieve the objects of the present disclosure, the cosmetic composition may be prepared and commercialized in any formulation selected from the above-listed formulations, and the present disclosure is not limited to the above examples. In addition, the cosmetic composition according to the present disclosure may be formulated using a conventional cosmetic preparation method.

Specifically, the cosmetic composition of the present disclosure may be prepared into any one formulation selected from the group consisting of a skin lotion, a skin softener, a skin toner, an astringent, a lotion, a milk lotion, a moisturizing lotion, a nourishing lotion, a massage cream, a nourishing cream, a moisturizing cream, a hand cream, an essence, a pack, a mask pack, a mask sheet, an exfoliating agent, a soap, a shampoo, a cleansing foam, a cleansing lotion, a cleansing cream, a body lotion, a body cleanser, an emulsion, a pressed powder, a loose powder, and an eye shadow.

The cosmetic composition of the present disclosure may further contain, in addition to the active ingredient (carvone or a salt thereof), other additives such as an excipient, a carrier, and the like, and common ingredients that are added to common skin cosmetics may be added to and mixed with the cosmetic composition in a needed amount.

In particular, the cosmetic composition of the present disclosure may further contain a transdermal penetration enhancer. The term “transdermal penetration enhancer” as used in the present disclosure refers to a composition that allows a desired component to penetrate vascular cells of the skin at a high absorption rate. Preferred examples of the transdermal penetration enhancer include, but are not limited to, other phospholipid components, liposomal components, and the like, which are used in lecithin cosmetics.

In addition, oil that may be mainly used as an oil phase component may be at least one selected from among vegetable oil, mineral oil, silicone oil, and synthetic oil. More particular examples of the oil include mineral oil, cyclomethicone, squalane, octyldodecyl myristate, olive oil, Vitis vinifera seed oil, macadamia nut oil, glyceryl octanoate, castor oil, ethylhexyl isononanoate, dimethicone, cyclopentasiloxane, sunflower seed oil, and the like.

In addition, to reinforce the emulsifying ability, 0.1 wt % to 5 wt % of a surfactant, a higher alcohol, or the like may be added. The surfactant may be a conventional surfactant such as a nonionic surfactant, an anionic surfactant, a cationic surfactant, an amphoteric surfactant, a phospholipid, or the like, and specific examples of the surfactant include sorbitan sesquinoleate, polysorbate 60, glyceryl stearate, lipophilic glyceryl stearate, sorbitan oleate, sorbitan stearate, DEA-cetyl phosphate, sorbitan stearate/sucrose cocoate, glyceryl stearate/polyethylene glycol-100 stearate, ceteareth-6 olivate, arachidyl alcohol/behenyl alcohol/arachidyl glucoside, polypropylene glycol-26-butes-26/polyethylene glycol-40 hydrogenated castor oil, and the like. The higher alcohol may be a C₁₂ to C₂₀ alcohol, for example, cetyl alcohol, stearyl alcohol, octyldodecanol, isostearyl alcohol, or the like, and these higher alcohols may be used alone or in combination of two or more thereof.

To adjust the viscosity or hardness of a water-phase component, 0.001 wt % to 5 wt % of at least one thickening agent selected from among carbomer, xanthan gum, bentonite, magnesium aluminum silicate, cellulose gum, dextrin palmitate, and the like may further be added.

In addition, the cosmetic composition according to the present disclosure may further contain, according to need, components, for example, a medicinal ingredient such as higher fatty acids, vitamins, or the like; a UV blocking agent; an antioxidant (butylhydroxyanisole, gallic acid propyl, erythorbic acid, tocopheryl acetate, butylated hydroxytoluene, or the like); a preservative (methylparaben, butylparaben, propylparaben, phenoxyethanol, imidazolidinyl urea, chlorphenethine, or the like); a colorant, a pH adjusting agent (triethanolamine, citric acid, sodium citrate, malic acid, sodium malate, fumaric acid, sodium fumarate, succinic acid, sodium succinate, sodium hydroxide, sodium monohydrogen phosphate, or the like); a moisturizing agent (glycerin, sorbitol, propylene glycol, butylene glycol, hexylene glycol, diglycerin, betaine, glycereth-26, methyl gluceth-20, or the like); a lubricant; and the like.

In addition, the cosmetic composition of the present disclosure may further contain an adjuvant for supplying an essential nutrient to the skin, preferably an adjuvant with natural flavor or cosmetic flavor, or medicinal herbs, but may contain any adjuvant without being limited to these examples.

The present disclosure also provides a topical skin composition for skin whitening containing carvone or a salt thereof as an active ingredient.

The term “topical skin composition” as used in the present disclosure is meant to include any substance that is generally used for external application to the skin. Non-limiting examples of formulations of the topical skin composition include plasters, lotions, liniments, liquids and solutions, aerosols, extracts, ointments, fluid extracts, emulsions, suspensions, capsules, creams, soft or hard gelatin capsules, patches, and sustained release formulations.

The topical skin composition according to the present disclosure may be a composition for parenteral administration formulated in a solid, semi-solid or liquid form by adding commercially available inorganic or organic carriers, excipients and diluents. The formulation for parenteral administration may be a transdermal dosage form selected from the group consisting of drops, ointments, lotions, gels, creams, patches, sprays, suspensions and emulsions, but is not limited thereto.

Carriers, excipients and diluents that may be contained in the topical skin composition include lactose, dextrose, sucrose, oligosaccharide, sorbitol, mannitol, xylitol, erythritol, maltitol, starch, gum acacia, alginate, gelatin, calcium phosphate, calcium silicate, cellulose, methyl cellulose, microcrystalline cellulose, polyvinyl pyrrolidone, water, methylhydroxybenzoate, propylhydroxybenzoate, talc, magnesium stearate, and mineral oil.

In the present disclosure, the content of carvone or a salt thereof is preferably 0.0001 to 20 wt % based on the total weight of the topical skin composition. If the content is more than 20 wt %, the stability of the prepared composition may decrease, and if the content of carvone or a salt thereof is less than 0.0001 wt %, the effect thereof is insignificant.

In the topical skin composition having each formulation, other ingredients other than the composition of the present disclosure may be appropriately selected and mixed by those skilled in the art without difficulty depending on the formulation or intended use of the other topical skin composition. In this case, when the composition is applied simultaneously with other ingredient, a synergistic effect may occur.

The present disclosure also provides a health functional food composition for skin whitening containing carvone or a food-acceptable salt thereof as an active ingredient.

The term “health functional food” as used in the present disclosure refers to foods prepared and processed in the form of tablets, capsules, powder, granules, a liquid, pills, or the like by using raw materials or ingredients having functionality useful for the human body. The term “functionality” as used herein refers to controlling nutrients for the structure and functions of the human body or providing useful effects for health care purposes, such as psychological effects. The health functional food of the present disclosure may be prepared using a method commonly used in the art, and may be prepared by adding raw materials and ingredients commonly added in the art. In addition, the formulation of the health function food is not particularly limited so long as it is recognized as a health functional food. The health functional food composition of the present disclosure uses a food as a raw material unlike generic drugs, and thus has no side effects that may occur during long-term administration thereof, is highly portable, and may be administered as an adjuvant for enhancing skin whitening.

The food functional food composition of the present disclosure may be prepared into any one formulation selected from the group consisting of tablets, granules, powder, capsules, a liquid solution, and pills.

The health functional food composition according to the present disclosure may be formulated in the form of powder, a liquid, tablets, soft capsules, granules, a tea bag, an instant tea, a drink, or the like by adding carvone or a salt thereof as an active ingredient. The content of carvone or a salt thereof as an active ingredient may be appropriately determined depending on the intended use thereof (use for prevention or amelioration). In general, the amount of the active ingredient (carvone or a salt thereof) contained in the health functional food composition may be 0.1 wt % to 20 wt % based on the total food weight. However, in the case of long-term administration for health care and hygiene purposes or for the purpose of adjusting health, the amount may be smaller than the lower limit of the above-described range. In addition, the health functional food composition according to the present disclosure may further contain, in addition to the active ingredient, other ingredients that may impart a synergistic effect to main effects within a range that does not impair desired main effects of the present disclosure, for example, a whitening improving compound such as vitamin C, or a natural substance such as a green tea extract, a paper mulberry extract, a licorice extract, a mulberry root extract, a betel nut extract, a Scutellaria baicalensis root extract, a wild ginseng extract, or the like.

The health functional food composition formulated in the above-described form may be directly added to a food or may be used in combination with other foods or food ingredients, and may be appropriately used according to a conventional method. Examples of foods include drinks, meats, sausages, bread, biscuits, rice cakes, chocolates, candies, snacks, confectionaries, pizzas, instant noodles, other noodles, gums, dairy products including ice creams, various kinds of soup, beverages, alcoholic drinks, vitamin complexes, and milk and dairy products, and all health functional foods in the ordinary sense are included.

When the health functional food composition of the present disclosure is a drink, the drink contains carvone or a salt thereof as an essential ingredient at the indicated ratio, and other ingredients used for the preparation of the drink are not particularly limited, and the drink may further contain various flavoring agents or natural carbohydrates, like general beverages. Examples of the natural carbohydrates include general sugars, for example, monosaccharides such as glucose, fructose, and the like; disaccharides such as maltose, sucrose, and the like; and polysaccharides such as dextrin, cyclodextrin, and the like, and sugar alcohols such as xylitol, sorbitol, erythritol, and the like. In addition to the above-described flavoring agents, natural flavoring agents, synthetic flavoring agents, and the like may be used as flavoring agents. The amount of the natural carbohydrate is generally about 1 g to about 20 g, preferably about 5 g to about 12 g, based on 100 ml of the composition of the present disclosure.

In addition, the health functional food composition of the present disclosure may contain various nutritional supplements, vitamins, minerals (electrolytes), flavoring agents such as a synthetic flavoring agent and a natural flavoring agent, colorants and fillers (cheese, chocolate, etc.), pectic acid and a salt thereof, alginic acid and a salt thereof, organic acids, protective colloid thickeners, a pH adjusting agent, a stabilizer, a preservative, glycerin, an alcohol, and a carbonating agent that is used in carbonated drinks. Additionally, the food composition of the present disclosure may contain fruit flesh that is used for the preparation of natural fruit juice, fruit juice beverages, and vegetable beverages. These ingredients may be used alone or in combination. The proportion of these additives is not so critical, but may generally range from 0.1 parts by weight to 20 parts by weight based on 100 parts by weight of the active ingredient (carvone or a salt thereof) of the present disclosure.

The present disclosure also provides a pharmaceutical composition for preventing or treating skin pigmentation containing carvone or a pharmaceutically acceptable salt thereof as an active ingredient.

The content of carvone or a salt thereof in the composition of the present disclosure is preferably 0.0001 wt % to 20 wt % based on the total weight of the composition. If the content is more than 20 wt %, the change in the effect of the active ingredient may be insignificant or the active ingredient may exhibit previously known toxicity, and if the content is less than 0.0001 wt %, the effect thereof is insignificant.

When carvone or a pharmaceutically acceptable salt thereof according to the present disclosure is used as a pharmaceutical, the pharmaceutical composition may further contain one or more active ingredients exhibiting the same or similar function. For example, the pharmaceutical composition may contain a known skin whitening ingredient. When the pharmaceutical composition further contains such a skin whitening ingredient, the skin whitening effect of the composition of the present disclosure may further be enhanced. When the above-described additional ingredients are used, skin safety upon the combined use thereof, easiness of formulation, and stability of the active ingredients may be considered. In one embodiment of the present disclosure, the pharmaceutical composition may further contain vitamin C or the like which is a whitening ingredient known in the art. The additional whitening improving ingredient may be contained in an amount of 0.0001 wt % to 10 wt % based on the total weight of the composition, and this content range may be adjusted according to requirements such as the effect of inhibiting tyrosinase activity, skin safety, and easiness of formulation of the compound of Formula 1 above.

In addition, the pharmaceutical composition for preventing or treating skin pigmentation according to the present disclosure may further contain a pharmaceutically acceptable carrier.

The pharmaceutically acceptable carrier may contain various components, such as a buffer, injectable sterile water, normal saline or phosphate buffered saline, sucrose, histidine, salts, and polysorbate.

The pharmaceutical composition of the present disclosure may be administered orally or parenterally, and may be administered in the form of over-the-counter drugs, for example, various formulations for oral and parenteral administration upon clinical administration. The pharmaceutical composition may be formulated using a commonly used diluent or excipient such as a filler, an extender, a binder, a wetting agent, a disintegrant, or a surfactant.

Examples of solid formulations for oral administration include tablets, pills, powder, granules, and capsules. Such solid formulations may be prepared by adding at least one excipient, for example, starch, calcium carbonate, sucrose, lactose, or gelatin, to the pharmaceutical composition of the present disclosure.

In addition to simple excipients, lubricants such as magnesium stearate and talc may also be used. Examples of liquid formulations for oral administration include suspensions, liquids and solutions, emulsions, and syrups. In addition to commonly used simple diluents such as water and liquid paraffin, various excipients, for example, a wetting agent, a sweetening agent, a fragrance, a preservative, and the like may be included.

Examples of formulations for parenteral administration include an aqueous sterile solution, a non-aqueous solution, a suspension, an emulsion, a freeze-dried preparation, and a suppository. Examples of a non-aqueous solvent and a suspending agent include propylene glycol, polyethylene glycol, vegetable oils such as olive oil, and injectable esters such as ethyl oleate. Examples of suppository bases include Witepsol, Macrogol, Tween 61, cacao butter, laurin fat, glycerogelatin, and the like.

When the pharmaceutical composition of the present disclosure contains an effective amount of carvone or a pharmaceutically acceptable salt thereof, it may provide a desired effect of preventing or treating skin pigmentation. The term “effective amount” as used in the present disclosure refers to the amount of a compound capable of preventing or inhibiting the production of skin pigmentation, or alleviating already produced pigment. The effective amount of carvone or a pharmaceutically acceptable salt thereof contained in the composition of the present disclosure may vary depending on the product type of the composition, a method of applying the compound to the skin, and the retention time on the skin. For example, when the composition is prepared into a drug for dermatological treatment of skin pigmentation, the prepared composition may contain carvone or a pharmaceutically acceptable salt thereof at a higher concentration than when the composition is prepared into a cosmetic product that is commonly applied to the skin.

The preferred dosage of the pharmaceutical composition according to the present disclosure may vary depending on the patient's condition and body weight, the severity of disease, the type of drug, the route of administration, and the duration of administration, and may be suitably determined by a person skilled in the art. To achieve the desired effect, however, the pharmaceutical composition of the present disclosure may be administered at a daily dose of 0.0001 to 2,000 mg/kg, preferably 0.001 to 2,000 mg/kg. The pharmaceutical composition may be administered once or several times a day. However, the dosage is not intended to limit the scope of the present disclosure in any way.

The present disclosure also provides a quasi-drug composition for preventing or ameliorating skin pigmentation comprising carvone or a pharmaceutically acceptable salt thereof as an active ingredient.

The term “quasi-drug” as used in the present disclosure refers to products that are less effective than pharmaceuticals, among products used for diagnosing, curing, ameliorating, alleviating, treating, or preventing diseases of humans or animals. For example, according to the Pharmaceutical Affairs Law, quasi-drugs exclude products for use as pharmaceuticals, and include products that are used for treating or preventing diseases of humans and animals, and products which minimally act on the human body or do not act directly on the human body.

The quasi-drug composition of the present disclosure is used for the purpose of preventing or ameliorating skin pigmentation, and formulations thereof are not particularly limited. For example, the quasi-drug composition may be a cosmetic composition having a formulation of a softening lotion, a nourishing lotion, a massage cream, a nourishing cream, a pack, a mask pack, a mask sheet, or a gel, or a skin adhesive-type cosmetic, and may also have a formulation for transdermal administration, such as a lotion, an ointment, a gel, a cream, a patch, or a spray.

In addition, the quasi-drug composition having each formulation may be mixed with other ingredients arbitrarily selected depending on the formulations or intended use of other quasi-drugs. The content of the active ingredient may be suitably determined depending on the intended use (use for inhibition or alleviation). For example, the quasi-drug composition may contain common adjuvants such as a thickener, a stabilizer, a solubilizer, vitamins, a pigment, and a flavoring agent, and a carrier.

The content of carvone or a salt thereof in the quasi-drug composition of the present disclosure is preferably 0.0001 wt % to 20 wt % based on the total weight of the quasi-drug composition. If the content is more than 20 wt %, color and stability of the prepared composition may deteriorate, and if the content is less than 0.0001 wt %, the effect thereof is insignificant.

In addition, in the quasi-drug composition having each formulation, ingredients other than the above-described essential ingredients may be appropriately selected and mixed by one of ordinary skill in the art without difficulty depending on the formulation or intended use.

Hereinafter, the present disclosure will be described in more detail with reference to examples. It will be obvious to those of ordinary skill in the art that these examples serve merely to illustrate the present disclosure, and the scope of the present disclosure is not construed as being limited by these examples.

### Examples: Evaluation of Melanogenesis Inhibitory Efficacy of Carvone Using B6F10 Melanocytes

1-1. Experimental Method

1) Experimental Materials and Cell Culture

Alpha-melanocyte stimulating hormone (α-MSH) and carvone used in this experiment were all purchased from Sigma Aldrich (St Louis, Mo., USA). B16F10 melanocytes were purchased from ATCC (Manassas, Va., USA), and the purchased cells were cultured using DMEM (Dulbecco's Modified Eagle's Media) containing 10% FBS (fetal bovine serum) in an incubator at 37° C. under 5% CO₂.

2) Measurement of Melanin Production in Cells

B6F10 melanocytes were cultured in DMEM containing 10% FBS at 37° C. under 5% CO₂, and then the cells were dispensed into a 6-well plate at a density of 1×10⁵ cells/well and attached thereto. As normal cells, cells not treated with α-MSH that induces melanogenesis were used, and as control cells, cells treated with α-MSH (100 nM) were used. The cells cultured under the above conditions were treated with α-MSH (100 nM) plus carvone (100 μM) and cultured for 48 hours.

After washing twice with PBS (phosphate buffered saline), the cells were harvested. The harvested cells were centrifuged at 1,000 rpm for 5 minutes, and then the color of the pellet was visually observed. Thereafter, the cell pellet was lysed by adding 200 μL of 1N NaOH solution containing 10% DMSO, and then the absorbance at 400 nm was measured with a microplate reader.

3) RNA Extraction and PCR Assay

Total RNA was extracted from cells using Trizol, and cDNA was synthesized by subjecting the extracted RNA sample to reverse transcription using an oligo dT primer and superscript reverse transcriptase (GIBCO BRL, Gaithersburg, Md., USA). Using the cDNA obtained through reverse transcription as a template and using the 5′ and 3′ flanking sequences of the gene cDNA to be amplified as primers, quantitative PCR was performed using iQ SYBR green supermix (Bio-Rad) and CFX Connect™ Real-Time PCR Detection System (Bio-Rad). The primer sequences used are shown in Table 1 below.

TABLE 1
Primer sequences used in PCR

| | | | Annealing | PCR |
|---|---|---|---|---|
| Gene | | | temperature | product |
| description | Primer | Sequence (5′→3′) | (° C.) | (bp) |
| Tyrosinase | F | CTCTGGGCTTAGCAGTAGGC | 55 | 164 |
| | | (SEQ ID NO: 1) | | |
| | R | GCAAGCTGTGGTAGTCGTCT | | |
| | | (SEQ ID NO: 2) | | |
| MITF | F | AGCGTGTATTTTCCCCACAG | 55 | 103 |
| (microphthalmia | | (SEQ ID NO: 3) | | |
| transcription | R | TAGCTCCTTAATGCGGTCGT | | |
| factor) | | (SEQ ID NO: 4) | | |
| GAPDH | F | CAAAATGGTGAAGGTCGGTG | 55 | 104 |
| (Glyceraldehyde | | (SEQ ID NO: 5) | | |
| dehydrogenase) | R | TTTGCCGTGAGTGGAGTCAT | | |
| 3-phosphate | | (SEQ ID NO: 6) | | |

1-2. Experimental Results

1) Melanogenesis Inhibitory Effect of Carvone in B6F10 Melanocytes

Melanin is synthesized in melanosomes, which are organelles in melanocytes, and is produced through a series of enzymatic reactions while melanocytes are activated when exposed to ultraviolet rays.

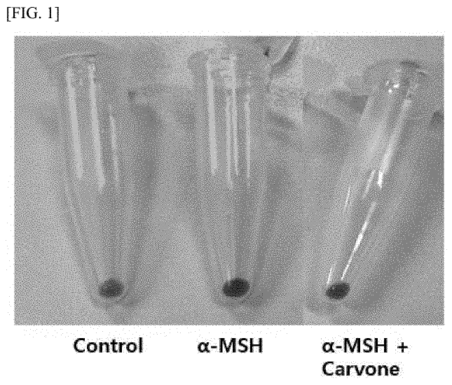

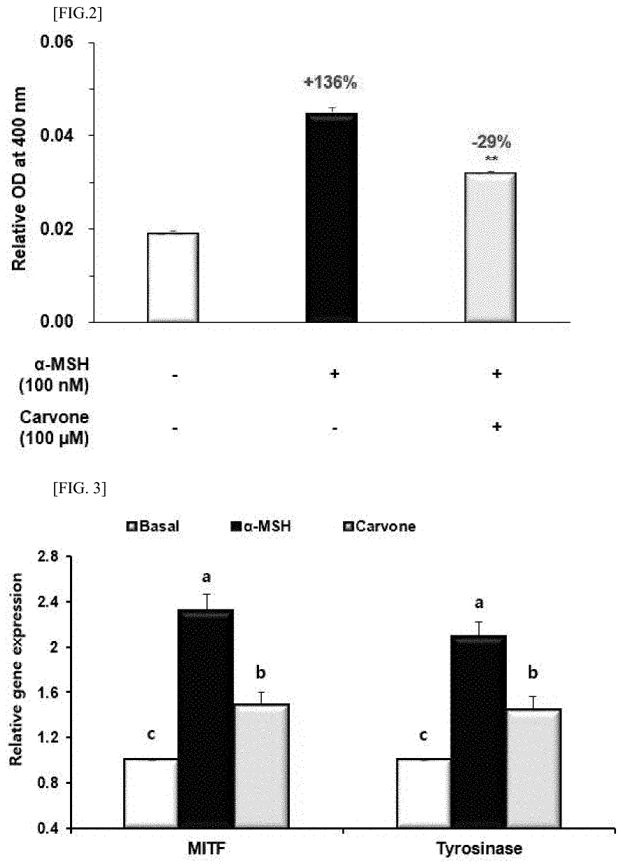

As a result of visually observing the pellet color of B6F10 melanocytes, it was confirmed that the pellet of the control cells treated with α-MSH turned blacker than the pellet of the normal cells not treated with α-MSH. It could be confirmed that the pellet color of the cells treated with α-MSH plus carvone was significantly paler than that of the control cells treated with only α-MSH (FIG. 1). As a result of lysing the cells and quantifying the color thereof, the melanin content of the control cells treated with α-MSH significantly increased by 136% compared to that of the normal cells not treated with α-MSH. Meanwhile, it was confirmed that the melanin content of the cells treated with α-MSH plus carvone significantly decreased by 29% compared to that of the control cells (FIG. 2). Thus, it can be seen that carvone has an excellent melanogenesis inhibitory effect.

2) Evaluation of Melanogenesis Inhibition Through Change in Gene Expression in B6F10 Melanocytes

Tyrosinase is well known to regulate the melanin synthesis rate by acting at the initial stage of melanin synthesis in melanocytes, and MITF is known to induce enzyme expression by binding to the E-box (CATGTG) present in the gene promoter of tyrosinase.

In order to examine whether the melanogenesis inhibitory effect of carvone would be associated with inhibition of expression of enzymes such as tyrosinase and regulation of expression of the transcription factor MITF, PCR assay was performed on B6F10 melanocytes. As a result, it was confirmed that the expressions of MITF and tyrosinase in the control cells treated with α-MSH significantly increased compared to those in the normal cells (basal). In addition, it was confirmed that the expressions of MITF and tyrosinase in the cells treated with α-MSH plus carvone significantly decreased by 36% and 31%, respectively, compared to those in the control cells (FIG. 3).

### Mode for Invention

Hereinafter, examples for preparation of pharmaceutical, food or cosmetic compositions containing the carvone as an active ingredient according to the present disclosure will be described, but these preparation examples serve merely to explain the present disclosure in detail and are not intended to limit the present disclosure. Using the composition components and composition ratios described below, the pharmaceutical, food or cosmetic compositions of Preparation Examples 1 to 3 were prepared according to conventional methods.

### [Preparation Example 1] Preparation of Cosmetics

1-1. Preparation of Skin Softener

Using the composition shown in Table 2 below, a skin softener containing carvone was prepared.

TABLE 2

| | Preparation Example 1-1 |
|---|---|
| Composition | (wt %) |
| Carvone | 0.1 |
| Ethanol | 10.0 |
| Polylauric acid | 1.0 |
| polyoxyethylene sorbitan | |
| Methyl paraoxybenzoate | 0.2 |
| Glycerin | 5.0 |
| 1,3-butylene glycol | 6.0 |
| Fragrance | q.s. |
| Pigment | q.s. |

1-2. Preparation of Nourishing Lotion

Using the composition shown in Table 3 below, a nourishing lotion containing carvone was prepared.

TABLE 3

| | Preparation Example |
|---|---|
| Composition | 1-2 (wt %) |
| Carvone | 0.05 |
| Veseline | 2.0 |
| Sorbitan sesquioleate | 0.8 |
| Polyoxyethylene ethyl oleate | 1.2 |
| Methyl paraoxybenzoate | q.s. |
| Propylene glycol | 5.0 |
| Ethanol | 3.2 |
| Carboxyvinyl polymer | 18.0 |
| Potassium hydroxide | 0.1 |
| Pigment | q.s. |
| Fragrance | q.s. |

1-3. Preparation of Nourishing Cream

Using the composition shown in Table 4 below, a nourishing cream containing carvone was prepared.

TABLE 4

| Composition | Preparation Example 1-3 (wt %) |
|---|---|
| Carvone | 0.2 |
| Stearic acid | 15.0 |
| Cetanol | 1.0 |
| Potassium hydroxide | 0.7 |
| Glycerin | 5.0 |
| Propylene glycol | 3.0 |
| Preservative | q.s. |
| Fragrance | q.s. |
| Purified water | To 100 |

1-4. Preparation of Pack

Using the composition shown in Table 5 below, a pack containing carvone was prepared.

TABLE 5

| Composition | Preparation Example 1-4 (wt %) |
|---|---|
| Carvone | 0.05 |
| Glycerin | 5.0 |
| Propylene glycol | 4.0 |
| Polyvinyl alcohol | 15.0 |
| Ethanol | 8.0 |
| Polyoxyethylene ethyl oleate | 1.0 |
| Methyl paraoxybenzoate | 0.2 |
| Pigment | q.s. |
| Fragrance | q.s. |

1-4. Preparation of Essence

Using the composition shown in Table 6 below, an essence containing carvone was prepared.

TABLE 6

| Composition | Preparation Example 1-5 (wt %) |
|---|---|
| Carvone | 0.2 |
| Propylene glycol | 10.0 |
| Glycerin | 10.0 |
| Sodium hyaluronate | 5.0 |
| aqueous solution (1%) | |
| Ethanol | 5.0 |
| Polyoxyethylene hydrogenated | 1.0 |
| castor oil | |
| Methyl paraoxybenzoate | 0.1 |
| Fragrance | q.s. |
| Purified water | To 100 |

### [Preparation Example 2] Preparation of Health Functional Foods

2-1. Preparation of Health Functional Food

Using the composition shown in Table 7 below, a health functional food containing carvone was prepared.

TABLE 7

| Composition | Preparation Example 2-1 | |
|---|---|---|
| Carvone | 1000 | mg |
| Vitamin mixture | q.s. | |
| Vitamin A acetate | 1.0 | mg |
| Vitamin E | 0.13 | mg |
| Vitamin B1 | 0.15 | mg |
| Vitamin B2 | 0.5 | mg |
| Vitamin B6 | 0.2 | μg |
| Vitamin B12 | 10 | mg |
| Vitamin C | 10 | μg |
| Biotin | 1.7 | mg |
| Nicotinic acid amide | 50 | μg |
| Folic acid | 0.5 | mg |
| Calcium pantothenate | q.s. | |
| Mineral mixture | q.s. | |
| Ferrous sulfate | 1.75 | mg |
| Zinc oxide | 0.82 | mg |
| Magnesium carbonate | 25.3 | mg |
| Monobasic potassium phosphate | 15 | mg |
| Dibasic potassium phosphate | 55 | mg |
| Potassium citrate | 90 | mg |
| Calcium carbonate | 100 | mg |
| Magnesium oxide | 24.8 | mg |

Although ingredients relatively suitable for use in health functional foods were mixed in a composition ratio of the vitamin and mineral mixture as an exemplary embodiment, the mixing ratio may be arbitrarily varied. In addition, the above-listed ingredients may be mixed together according to a conventional method of preparing a health functional food, and then prepared into granules, which may then be used for the preparation of a health functional food composition according to a conventional method.

2-2. Preparation of Health Drink

Using the composition shown in Table 8 below, a health drink containing carvone was prepared.

TABLE 8

| Composition | Preparation Example 2-2 (mg) | |
|---|---|---|
| Carvone | 1000 | mg |
| Citric acid | 1000 | mg |
| Oligosaccharide | 100 | g |
| Plum concentrate | 2 | g |
| Taurine | 1 | g |

| | |
|---|---|
| Purified water | q.s. |

The above-listed ingredients were mixed together according to a conventional method of preparing a health drink, the mixture was heated and stirred at 85° C. for about 1 hour, and the resulting solution was filtered. The filtrate was collected in a 2-L sterilized container and then sealed, sterilized, cold-stored, and then used for the preparation of a heath drink composition according to the present disclosure. Although ingredients relatively suitable for use in favorite beverages were mixed together in the above-described composition ratio as an exemplary example, the mixing ratio may be arbitrarily varied depending on local and national preferences such as demand classes, demand countries, the intended use, and the like.

2-3. Preparation of Tablets

Using the composition shown in Table 9 below, tablets containing carvone was prepared by tableting the composition according to a conventional tablet preparation method.

TABLE 9

| Composition | Preparation Example 2-3 (mg) |
|---|---|
| Carvone | 100 mg |
| Corn starch | 100 mg |
| Lactose | 100 mg |
| Magnesium stearate | 2 mg |

2-4. Preparation of Capsules

Using the composition shown in Table 10 below, a capsule containing carvone was prepared by filling a gelatin capsule with the composition according to a conventional capsule preparation method.

TABLE 10

| Composition | Preparation Example 2-4 (mg) |
|---|---|
| Carvone | 100 mg |
| Corn starch | 100 mg |
| Lactose | 100 mg |
| Magnesium stearate | 2 mg |

2-5. Preparation of Pills

Using the composition shown in Table 11 below, pills were prepared according to a conventional pill preparation method so that the weight of each pill was 4 g.

TABLE 11

| Composition | Preparation Example 2-5 (mg) |
|---|---|
| Carvone | 1.0 g |
| Lactose | 1.5 g |
| Glycerin | 1.0 g |
| Xylitol | 0.5 g |

2-6. Preparation of Granules

Using the composition shown in Table 12 below, granules were prepared by adding 100 mg of 30% ethanol to the composition, drying the resulting mixture dried at 60° C. to form granules, and then filling a bag with the granules.

TABLE 12

| Composition | Preparation Example 2-6 (mg) |
|---|---|
| Carvone | 150 mg |
| Soybean extract | 50 mg |
| Glucose | 200 mg |
| Starch | 600 mg |

### Preparation Example 3

Preparation of Topical Skin Ointment

Using the composition shown in Table 13 below, a topical skin ointment containing carvone was prepared.

TABLE 13

| Composition | Preparation Example 3 (wt %) |
|---|---|
| Carvone | 0.5 |
| Diethyl sebacate | 8.0 |
| Spermaceti | 5.0 |
| Polyoxyethylene oleyl | 6.0 |
| ether phosphate | |
| Sodium benzoate | q.s. |

1. A method for whitening skin of a subject in need thereof, comprising administering or taking a composition containing carvone or a cosmetically acceptable salt thereof as an active ingredient to the subject.

2. The method of claim 1, wherein the whitening comprises inhibition of expression of microphthalmia transcription factor (MITF) or tyrosinase.

3. The method of claim 1, wherein the whitening comprises inhibition of melanogenesis induced by melanocyte-stimulating hormone (α-MSH).

4. The method of claim 1, wherein the composition is a cosmetic composition, foodstuff, food additive, nutraceutical composition, or dietary supplement.

5. The method of claim 4, wherein the composition is prepared into any one formulation selected from the group consisting of tablets, granules, powders, capsules, liquid solutions, and pills.

6. (canceled)

7. (canceled)

8. A method for preventing or treating skin pigmentation of a subject in need thereof, comprising a step of administering or taking a pharmaceutical composition containing a carvone or a pharmaceutically acceptable salt thereof as an active ingredient to the subject.

9. (canceled)


SEQUENCE LISTING (6 sequences)

SEQ ID NO: 1 - DNA, 20 bp, Artificial Sequence
  Tyrosinase_F primer
ctctgggcttagcagtaggc

SEQ ID NO: 2 - DNA, 20 bp, Artificial Sequence
  Tyrosinase_R primer
gcaagctgtggtagtcgtct

SEQ ID NO: 3 - DNA, 20 bp, Artificial Sequence
  MITF_F primer
agcgtgtattttccccacag

SEQ ID NO: 4 - DNA, 20 bp, Artificial Sequence
  MITF_R primer
tagctccttaatgcggtcgt

SEQ ID NO: 5 - DNA, 20 bp, Artificial Sequence
  GAPDH_F primer
caaaatggtgaaggtcggtg

SEQ ID NO: 6 - DNA, 20 bp, Artificial Sequence
  GAPDH_R primer
tttgccgtgagtggagtcat